(19) United States
(12) Patent Application Publication
(10) Pub. No.: US 2011/0004058 A1
(43) Pub. Date: Jan. 6, 2011

(54) Controllable Endoscope

(75) Inventors: Katsumi Oneda, Alpine, NJ (US); Ron Hadani, Cresskill, NJ (US)

Correspondence Address: MARTIN D. MOYNIHAN d/b/a PRTSI, INC., P.O. BOX 16446, ARLINGTON, VA, 22215, US

(73) Assignee: VISION - SCIENCES INC., Orangeburg, NY (US)

(21) Appl. No.: 12/223,313

(22) PCT Filed: Jan. 30, 2007
(86) PCT No.: PCT/IL2007/000116

§ 371 Date: Sep. 16, 2010

Related U.S. Application Data
(60) Provisional application No. 60/763,267, filed on Jan. 30, 2006.

(51) Int. Cl. A61B 1/04

(52) U.S. Cl. 600/109

(57) ABSTRACT

An endoscope including an outer elongate tube and an inner shaft that runs through the outer elongate tube and can be advanced independently of the outer tube. In addition, the endoscope includes an imaging capsule including at least one image capturing unit, mounted on a distal end of the inner shaft. The imaging capsule has a largest cross-section area smaller than the cross section area of the outer elongate tube.

### RELATED APPLICATIONS

This application claims the benefit under 119(e) of U.S. provisional application 60/763,267, filed Jan. 30, 2006, the disclosure of which is incorporated herein by reference.

### FIELD OF THE INVENTION

The present invention relates to minimally invasive endoscopes and particularly to colonscopes.

### BACKGROUND OF THE INVENTION

Minimally invasive procedures are used to access many body cavities for visual examination, tissue retrieval (biopsy) and treatment. The colon is relatively hard to access due to its long length and the sharp curves defined along its length.

One method for accessing the colon is use of swallowable capsules that carry video cameras. The swallowable capsules suffer from problems in maneuvering to exact locations. In addition, although swallowable capsules that collect biopsies have been described, their operation in collecting biopsies is considered problematic if operable at all.

U.S. Pat. No. 6,468,203 to Belson, the disclosure of which is incorporated herein by reference, describes a steerable endoscope for maneuvering through the colon.

U.S. Pat. No. 6,162,171 to Ng et al., the disclosure of which is incorporated herein by reference, describes a robotic endoscope including a plurality of segments connected together by a plurality of flexible articulated joints.

U.S. patent publication 2004/0186349 to Ewers et al., the disclosure of which is incorporated herein by reference, describes a colonscope having an outer sheath which can be inflated at its distal end, so as to engage the walls of the colon.

Another method of inserting a medical device into the colon is described in U.S. Pat. Nos. 207,932 to Alvord, patented Sep. 10, 1878, 5,601,537 to Frassica, 5,871,475 to Frassica, 5,989,230 to Frassica and US patent publication 2002/0045855 to Frassica, the disclosures of all of which are incorporated herein by reference. The method described in these patent documents includes using a medical probe, such as a colonscope, which has a screw threading on its distal end. The surgeon rotates a proximal end of the medical probe for advancement of the probe into the patient.

The endoscopes of the above listed patents are believed to have good maneuverability but slow advancement and limited view at their distal end.

Faster advancement is achieved, for example, by self-propelled units, such as described in U.S. Pat. No. 6,702,734 to Kim et al. and U.S. Pat. No. 6,866,626 to Long et al., the disclosures of which are incorporated herein by reference. The maneuverability of these self propelled units through sharp bends in the colon is, however, problematic.

In some cases, additional tools are used to aid in delivery of an endoscope.

U.S. Pat. No. 6,869,393 to Butler, the disclosure of which is incorporated herein by reference, describes an insertion device for aiding introduction of an overtube into a colon over a previously inserted colonscope.

PCT publication WO 2006/072928, published Jul. 13, 2006, the disclosure of which is incorporated herein by reference, describes a gastrointestinal tool with a bore for an elongate guiding element. Delivery of the tool on the guiding element is claimed to allow faster insertion of the tool.

Separate insertion of the guide wire requires insertion of an additional element having the only utility of aiding insertion of another tool.

U.S. Pat. No. 5,653,677 to Okada et al., the disclosure of which is incorporated herein by reference, describes an electronic endoscope having an imaging unit separable from a distal end of the endoscope. In one embodiment of the '677 patent, a wire connects the separable imaging unit to a distal end of the endoscope, for retrieving the imaging unit back into a slot on the distal end of the endoscope. The wire includes a signal line which is used to transfer acquired image data from the detachable imaging unit to the endoscope. The imaging unit is pushed ahead from the distal end of the endoscope by air or water supply.

U.S. Pat. Nos. 6,736,773 and 7,004,900 to Wendlandt et al., patented May 18, 2004 and Feb. 28, 2006, respectively, the disclosures of which are incorporated herein by reference, describe an endoscope with a vision head mounted on an extension arm for moving the vision head away from the endoscope.

U.S. patent publication 2003/0167007 to Belson, the disclosure of which is incorporated herein by reference, describes a colonscope with a spectroscopic examination unit extending therefrom. The spectroscopic examination unit is rotatable relative to the colonscope.

### SUMMARY OF THE INVENTION

An aspect of some embodiments of the present invention relates to an endoscopic shaft carrying at its distal end an imaging capsule which is adapted for advancement on its own along a substantial length of a body lumen.

In some embodiments of the invention, the imaging capsule has a longer axial dimension than an end-to-end length (referred to herein as diameter) of its cross-section. Alternatively or additionally, the imaging capsule has a cross section area substantially smaller than the cross section area of an endoscope (e.g., colonscope) with which it operates. In some embodiments of the invention, the imaging capsule is adapted to pass through a channel (e.g., a working channel) of the endoscope with which it operates. For example, the imaging capsule optionally has a maximal diameter of less than 3.5 millimeters or even less than 3 or 2.5 millimeters, at leased in a collapsed state.

Using an imaging capsule with a small diameter may allow fast advancement of the imaging capsule along non-curved sections of the colon.

In some embodiments of the invention, in advancement within the colon, the shaft with the imaging capsule is first advanced and then the endoscope is advanced on the shaft, which serves as a guide wire for the endoscope. The endoscope optionally carries apparatus which it is hard to mount on an imaging capsule with a small diameter, such as one or more working channels, fluid (e.g., air and/or water) channels, biopsy taking apparatus and/or an articulation mechanism.

In some embodiments of the invention, the imaging capsule comprises at least one camera with a field of view covering the advancement direction of the imaging capsule. Alternatively or additionally, the imaging capsule includes at least one camera with a view in a direction substantially opposite the direction of advancement of the imaging capsule (i.e., in the proximal direction), i.e., towards the endoscope. Further alternatively or additionally, the imaging capsule includes one or more cameras at an angle to a main axis of the endoscope, for example with a view orthogonal to the main axis of the endoscope.

In some embodiments of the invention, the endoscope includes an irrigation system adapted for cleaning a viewing window of the imaging capsule and/or the walls of the colon. Optionally, the irrigation system is adapted for cleaning the viewing window of the imaging capsule when the imaging capsule is within a pocket at a distal end of the endoscope, after an outer sleeve of the endoscope is advanced past the camera or the imaging capsule is retracted toward the sleeve.

The imaging capsule defines, in some embodiments of the invention, an indent which coincides with a working channel of the endoscope or otherwise allows passage of working tools and/or fluids passing through the working channel. Alternatively or additionally, the imaging capsule has a smaller cross section area than the endoscope, allowing free access to the working channel.

In some embodiments of the invention, the imaging capsule has a balloon mounted thereon, which is inflatable for anchoring the imaging capsule within the patient. The anchoring may allow faster delivery of the endoscope along the shaft of the imaging capsule. Optionally, the balloon is peripheral to the portion of the imaging capsule through which images are acquired, such that inflating the balloon does not affect the acquiring of images. Alternatively, a camera or fiber bundle of the imaging capsule is mounted on the balloon, such that inflating the balloon can be used to adjust a direction of image acquisition. For example, the imaging capsule may have a collapsed state in which it is adapted to be advanced through a body lumen such as the intestine, and an imaging state, e.g., an inflated state, in which it is used for imaging and/or for anchoring.

An aspect of some embodiments of the present invention relates to an elongate invasive probe, such as an endoscope or a catheter, comprising at least two separate portions rotatable relative to each other, and having outer surfaces adapted to contact patient tissue when the invasive probe is inserted into the patient. At least one of the portions has a threaded screw structure, optionally a helical threading, on its outer surface and is configured to be rotated by a human controller so as to cause advancement of the invasive probe or a portion thereof within body channels.

In some embodiments of the invention, the threaded portion of the invasive probe is connected through an internal shaft to a proximal end of the invasive probe, optionally to the proximal end of the patient. The internal shaft may be rotatable by a physician and/or a motor connected to a proximal end of the internal shaft is optionally used to rotate the threaded portion. Alternatively or additionally, a miniature motor is located within the invasive probe, adapted to rotate portions of the invasive probe relative to each other.

In an exemplary embodiment of the invention, a plurality of separately rotatable portions of the invasive probe have threadings on their external surface. There is therefore provided in accordance with some embodiments of the present invention, a colonscope or other endoscope including an outer elongate tube, an inner shaft that runs through the outer elongate tube and can be advanced independently of the outer tube and an imaging capsule including at least one image capturing unit, mounted on a distal end of the inner shaft, the imaging capsule having a largest cross-section area smaller than the cross section area of the outer elongate tube.

Optionally, the outer tube comprises a slot adapted to receive at least half of the imaging capsule, on its distal end. Optionally, the slot is adapted to receive the imaging capsule in its entirety. Optionally, when the outer tube includes at least one channel adapted to direct fluids at the imaging capsule, when the imaging capsule is in the slot.

Optionally, the outer tube comprises an articulation section which can be controllably articulated. Optionally, the inner shaft does not have a mechanism for controllable articulation. Optionally, the inner shaft has different levels of flexibility over its length.

Optionally, the inner shaft has a flexibility which decreases toward the imaging capsule. Optionally, the image capturing unit comprises a CCD. Optionally, the imaging capsule comprises at least one image capturing unit with a view directed proximally toward the outer tube. Optionally, the imaging capsule has a largest diameter at least twice the diameter of the inner shaft. Optionally, the outer elongate tube defines at least one working channel running along its length. Optionally, an extent of axial movement of the inner elongate tube within the outer elongate tube is limited. Optionally, the imaging capsule has a larger cross section than the inner elongate tube, which prevents it from being retracted proximally through the outer elongate tube. Optionally, the imaging capsule is adapted to pass through a channel of the outer tube.

Optionally, the imaging capsule has a collapsed state in which it can pass through a channel of the outer tube and an expanded state in which it cannot pass through a channel of the outer tube. Optionally, an axial extent of a portion of the imaging capsule having a cross-section area larger than a cross section area of the inner shaft is greater than a diameter of the imaging capsule.

Optionally, an axial extent from a most proximal camera of the imaging capsule to its distal end is greater than a diameter of the imaging capsule. Optionally, the imaging capsule has a cross-section diameter of less than 3.5 millimeters. Optionally, the imaging capsule comprises a balloon. Optionally, the balloon is adapted to expand radially, without displacing an imaging direction of the imaging capsule.

There is further provided in accordance with an exemplary embodiment of the invention, a first portion comprising a tube defining an outer surface adapted to contact internal body tissue, a second portion comprising an outer surface adapted to contact internal body tissue, the second portion being adapted to rotate relative to the first portion and a helical threading on the outer surface of the second portion, adapted to advance the second portion within a body cavity when the second portion is rotated relative to the body portion.

Optionally, the probe includes a motor adapted to rotate the second portion. Optionally, the second portion is close to a distal end of the medical probe. Optionally, the probe includes an internal shaft extending through the first portion and connecting the second portion to a proximal end of the medical probe.

There is further provided in accordance with an exemplary embodiment of the invention, a method of advancing an endoscope within a patient, comprising inserting a shaft having an imaging capsule at its distal end to a body lumen of a patient; and advancing an outer tube, having a cross section larger than the cross section of the shaft, over the shaft, into the body lumen.

Optionally, the imaging capsule has a cross section area greater than the cross section area of the shaft. Optionally, the imaging capsule has a cross section area substantially equal to the cross section area of the shaft. Optionally, the outer tube comprises an endoscope.

Optionally, the outer tube comprises an articulation system and the shaft does not comprise an articulation system. Optionally, the imaging capsule comprises at least one camera mounted thereon. Optionally, inserting the shaft comprises inserting together with the outer tube. Optionally, inserting the shaft comprises inserting through a channel of the outer tube, after the outer tube was inserted separately into the patient.

Optionally, advancing the outer tube over the shaft comprises advancing the outer tube with a channel of the outer tube riding over the shaft. Alternatively or additionally, advancing the outer tube over the shaft comprises connecting a sleeve to the outer tube and passing the sleeve over the shaft.

### BRIEF DESCRIPTION OF THE DRAWINGS

Exemplary non-limiting embodiments of the invention will be described with reference to the following description of the embodiments, in conjunction with the figures. Identical structures, elements or parts which appear in more than one figure are preferably labeled with a same or similar number in all the figures in which they appear, and in which:

FIG. 1 is a schematic illustration of a colonscope, in accordance with an exemplary embodiment of the invention;

FIG. 2 is a cross-section view of the colonscope of FIG. 1, toward its distal end, in accordance with an exemplary embodiment of the invention;

FIG. 3 is a cross-sectional view of the colonscope of FIG. 1, in an intermediate section, in accordance with an exemplary embodiment of the invention;

FIGS. 4A-4C are schematic illustrations of an imaging capsule and an inner shaft connected thereto, in accordance with an exemplary embodiment of the invention;

FIG. 5 is a flowchart of acts performed in a medical procedure using a colonscope, in accordance with an exemplary embodiment of the invention;

FIG. 6A is a schematic illustration of an imaging unit in a collapsed state, in accordance with another exemplary embodiment of the invention;

FIG. 6B is a schematic illustration of the imaging unit of FIG. 6A in an expanded state, in accordance with another exemplary embodiment of the invention;

FIG. 6C is a cross section view of the imaging unit of FIG. 6A in an expanded state, in accordance with another exemplary embodiment of the invention;

FIG. 6D is a schematic illustration of a foldable tube, in accordance with another exemplary embodiment of the invention;

FIGS. 7A and 7B are side and back views of an imaging unit, in accordance with still another exemplary embodiment of the invention;

FIG. 7C illustrates an inner tube of the imaging unit of FIGS. 7A and 7B in a rest state, in accordance with an exemplary embodiment of the invention;

FIG. 8 is a schematic illustration of use of an imaging capsule and shaft as a monorail for an insertion tube of an endoscope, in accordance with an exemplary embodiment of the invention;

FIG. 9 is a schematic illustration of a colonscope, in accordance with another exemplary embodiment of the invention; and

FIG. 10 is a schematic cross sectional view of an endoscope, in accordance with still another exemplary embodiment of the invention.

### DETAILED DESCRIPTION OF EXEMPLARY EMBODIMENTS

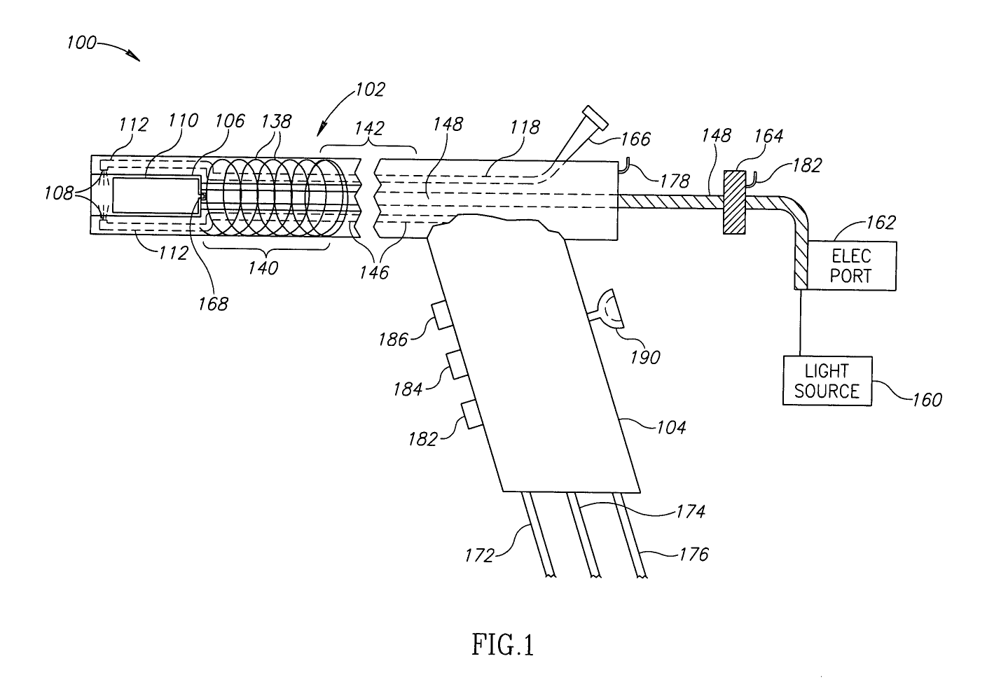

FIG. 1 is a schematic illustration of a colonscope 100, in accordance with an exemplary embodiment of the invention. Colonscope 100 includes an elongate tube 102 and a control handle 104. In some embodiments of the invention, elongate tube 102 is divided axially into three major sections. At its distal end, elongate tube 102 defines a slot 106 in which an imaging capsule 110 resides. One or more outlets 108 with respective distal channels 112 may be used to eject water and/or air on a distal end of imaging capsule 110, for example in order to clean a viewing window of the imaging capsule. An articulation section 140 located adjacent slot 106 can be used for steering elongate tube 102. A proximal section 142 is located between articulation section 140 and handle 104.

### Distal End

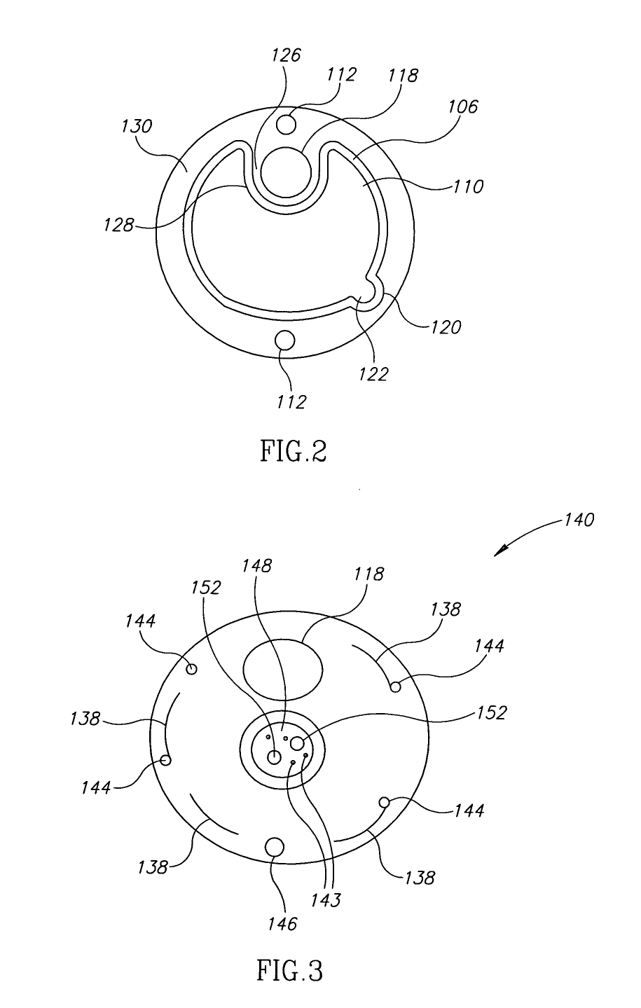

FIG. 2 is a cross-sectional view of elongate tube 102 close to its distal end, in accordance with an exemplary embodiment of the invention. Near its distal end, elongate tube 102 includes an outer sleeve 130, which defines internal slot 106. Optionally, distal channels 112 and/or a working channel 118 are defined within outer sleeve 130. In some embodiments of the invention, outer sleeve 130 has a generally annular shape in which an imaging capsule 110 with a generally circular cross-section is received. In some embodiments of the invention, outer sleeve 130 defines one or more indents and/or bulges, with respective bulges and/or indents on imaging capsule 110, in order to maintain the alignment of imaging capsule 110 relative to outer sleeve 130. Optionally, working channel 118 is located in an inner bulge 126 of outer sleeve 130, so as to accommodate a relatively large diameter for working channel 118. Imaging capsule 110 optionally has a respective indent 128, which matches bulge 126 and aligns the capsule within slot 106. Thus, working channel 118 can be used even when imaging capsule 110 is within slot 106. Alternatively or additionally, a bulge 122 in imaging capsule 110 matches an indent 120 in sleeve 130. Further alternatively or additionally, working channel 118 does not extend through slot 106, such that the working channel can be used only when imaging capsule 110 is not within the slot.

As shown in FIG. 1, imaging capsule 110 fits entirely in slot 106, such that the distal end of imaging capsule 110 does not extend beyond the distal end of outer sleeve 130, when the imaging capsule is entirely within the slot. Alternatively, imaging capsule 110 only partially fits into slot 106. Optionally, at least 20%, 40% or even at least 50% of the length of imaging capsule 110 fit in slot 106. In some embodiments of the invention, less than 80%, less than 60% or even less than 40% of the length of imaging capsule 110 fit into slot 106.

In other embodiments of the invention, colonscope 100 does not include a slot for receiving imaging capsule 110, but rather has a mechanism for attaching the imaging capsule to the distal end of elongate tube 102, for example an electromagnet and/or a mechanical lock. Optionally, any other attaching mechanism and/or imaging capsule shape and structure may be used, such as using any of the features described in U.S. Pat. No. 5,653,677, U.S. Pat. No. 6,632,171 and/or U.S. patent publication 2001/0051766, the disclosures of which are incorporated herein by reference. In some embodiments of the invention, no attachment method is used and imaging capsule 110 is merely placed adjacent the distal end of elongate tube 102 when it is desired to advance imaging capsule and elongate tube 102 together.

When imaging capsule 110 is within slot 106, outlets 108 (FIG. 1) are optionally directed at a viewing window of imaging capsule 110. In order to clean a viewing window of imaging capsule 110, a physician retracts the imaging capsule into slot 106 and a fluid (e.g., water) is caused to flow through distal channels 112 and outlets 108 at imaging capsule 110.

In some embodiments of the invention, imaging capsule 110 has a cross-sectional area which is less than 80%, less than 60% or even less than 40% of the cross sectional area of elongate tube 102, allowing easier and/or faster advancement within body lumens, such as the intestine. In an exemplary embodiment of the invention, the cross section of imaging capsule 110 has a diameter of less than 4 millimeters, less than 3.5 millimeters or even less than 3 millimeters. Optionally, the diameter of the cross section of imaging capsule 110 is smaller than the axial length of the imaging capsule, possibly even smaller than the length of slot 106.

### Cross Section of Tube

Reference is also made to FIG. 3, which is a cross-sectional view of elongate tube 102 of FIG. 1 in articulation section 140, in accordance with an exemplary embodiment of the invention. In articulation section 140, elongate tube 102 includes an articulation coil 138 which is controlled through wires 144 from handle 104. FIG. 3 also shows working channel 118 and an irrigation channel 146 which connects to the one or more distal channels 112. An inner shaft 148, which is axially movable relative to elongate tube 102, connects imaging capsule 110 to handle 104. In some embodiments of the invention, inner shaft 148 includes one or more optical fibers 152 which lead light to imaging capsule 110, in order to illuminate areas of the colon being viewed, and/or which lead images acquired by imaging capsule 110 to a proximal end of colonscope 100. Alternatively or additionally, inner shaft 148 includes wires 143 which lead electrical power and/or data signals between handle 104 and imaging capsule 110, for example for leading electrical image data from a camera mounted on the imaging capsule to a point outside colonscope 100. In some embodiments of the invention, at least some of wires 143 lead clock signals, used by the camera and/or by video sampling apparatus, control signals and/or power to imaging capsule 110. In other embodiments of the invention, wires 143 do not pass through inner shaft 148, for example when a wireless transmitter is used to convey image data collected by imaging capsule 110 to a control station outside the patient.

In some embodiments of the invention, as discussed above, working channel 118 is fixed to elongate tube 102. Alternatively, working channel 118 is axially movable relative to elongate tube 102. While only a single working channel 118 is shown, in some embodiments of the invention colonscope 100 includes a plurality of working channels.

Referring back to FIG. 1, inner shaft 148 optionally connects at its distal end to imaging capsule 110 and extends proximally behind handle 104, where it connects to a light source 160 and/or an electrical port 162. Electrical port 162 is used to exchange control signals and sensed data with a computer or other control unit (not shown). In some embodiments of the invention, inner shaft 148 has a control knob 164 which controls the movement of inner shaft 148. Alternatively or additionally, any other controller may be used to aid and/or control the advancement and/or retraction of inner shaft 148, along with imaging capsule 110, relative to elongate tube 102.

Optionally, imaging capsule 110 is releasably connected to inner shaft 148, for example through a quick connect mechanism 168. The releasable connection of imaging capsule 110 is optionally used to disconnect the imaging capsule during cleaning of colonscope 100 and/or during other maintenance tasks. In some embodiments of the invention, imaging capsule 110 is attachable to a plurality of inner shafts, for example allowing use of a single imaging capsule 110 with a plurality of disposable inner shafts 148. Optionally, imaging capsule 110 may be used with inner shafts 148 of different lengths and/or widths.

Alternatively or additionally, imaging capsule 100 may be disconnected from inner shaft 148 within the patient, for example in order to allow a desired orientation of imaging capsule 110, which is not achievable when connected to inner shaft 148. In accordance with this alternative, imaging capsule 110 optionally includes a camera mounted thereon and a large memory for storing acquired images until imaging capsule 110 is reconnected to inner shaft 148 and the acquired images can be transmitted through wires 143 within inner shaft 148 to a control unit outside the patient. Alternatively or additionally, imaging capsule 110 includes a wireless transmitter for transmitting the images to outside the patient. It is noted that the use of a large memory and/or a wireless transmitter in imaging capsule 110 is not limited to this alternative, and may be used with substantially any embodiment of the invention.

In those embodiments in which imaging capsule 110 is detachable from inner shaft 148 within the patient, imaging capsule 110 optionally includes a self propagation mechanism, such as a flagellum driving member, a caterpillar driving unit or any other self-propelling means. Alternatively or additionally, imaging capsule 110 may be driven from outside the patient, for example using magnetic coupling.

Furthermore, in those embodiments in which imaging capsule 110 is detachable from inner shaft 148 within the patient, colonscope 100 optionally includes mechanisms which allow fast recombination with the inner shaft 148. For example, imaging capsule 110 may be connected to a thin and strong wire, which is pulled to bring the imaging capsule back into slot 106 and to connection with inner shaft 148.

Optionally, colonscope 100 has a dedicated channel for inner shaft 148, which is not used for other tasks. Alternatively, the channel used for inner shaft 148 may be used for other tasks. For example, after positioning imaging capsule 110, inner shaft 148 may be disconnected from the capsule and removed from the channel, which can then be used for other tasks. Alternatively or additionally, the channel may be used for other tasks, while inner shaft 148 is in it.

In some embodiments of the invention, imaging capsule 110 is delivered through a working channel of an endoscope not necessarily adapted for use with the imaging capsule. Optionally, in these embodiments, imaging capsule 110 has a relatively small cross-section, at least when it is in a delivery state. Optionally, after using imaging capsule 110 for viewing the capsule and shaft 148 may be removed from the patient and the working channel may be used for other tasks. Alternatively or additionally, as mentioned above, only inner shaft 148 is removed and/or the channel is used for other tasks with shaft 148 in the channel.

### Handle

At its proximal end, working channel 118 is optionally connected to a biopsy port 166, through which tools (e.g., biopsy forceps, a wire snare), and fluids may be passed to the distal end of working channel 118. In some embodiments of the invention, biopsy port 166 is also used for applying suction to the distal end of working channel 118. Alternatively or additionally, working channel 118 connects through handle 104 to a suction line 172, which in turn is connected to a suction source (not shown). In a similar manner, irrigation channel 146 is optionally connected through handle 104 to a water line 174 and/or an air line 176. In some embodiments of the invention, a suction button 182, a water button 184 and an air button 186 control passage of fluids through suction line 172, water line 174 and air line 176, respectively. Alternatively or additionally to air line 176 and/or water line 174 being connected to irrigation channel 146, air line 176 and/or water line 174 are controllably connected to working channel 118.

An articulation joystick 190 on handle 104 optionally controls articulation of elongate tube 102, for example through wires 144.

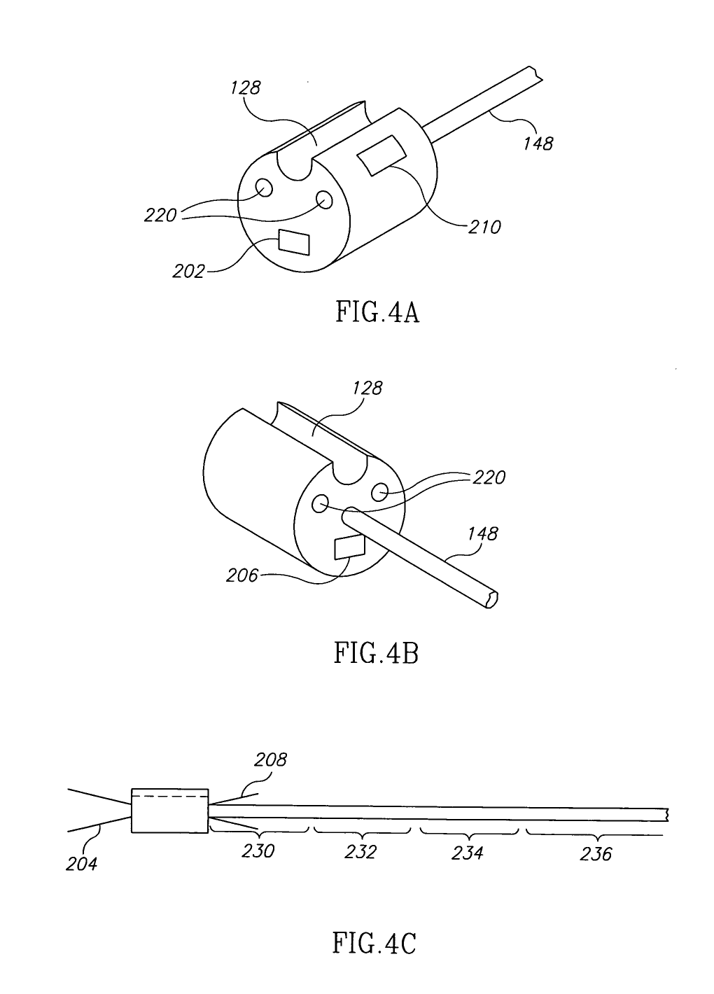

FIGS. 4A-4C are schematic illustrations of imaging capsule 110 and inner shaft 148, in accordance with an exemplary embodiment of the invention. Imaging capsule 110 optionally includes at least one front camera 202 (FIG. 4A, shown schematically by a rectangle) pointing distally with a forward view 204 (FIG. 4C). Optionally, a rear camera 206 (FIG. 4B) provides a rear view 208 (FIG. 4C). In some embodiments of the invention, imaging capsule 110 also has one or more side cameras 210. Cameras 202, 206 and 210 optionally include charge-coupled devices (CCDs), CMOS cameras or any other miniature cameras suitable for operation in an endoscope. Alternatively or additionally to cameras, viewing optics passing through inner shaft 148 are used to lead images to a camera or display unit positioned on or near handle 104 or otherwise outside the patient. In some embodiments of the invention, cameras 202, 206 and 210 include suitable lenses as is known in the art. The cameras may be for example as described in any of the above mentioned patents and/or in U.S. Pat. No. 5,604,531 to Iddan et al., the disclosure of which is incorporated herein by reference.

Imaging capsule 110 optionally includes illumination ports 220 which illuminate tissue being imaged. Illumination ports 220 optionally provide light passed through inner shaft 148. Alternatively or additionally, one or more of illumination ports 220 comprises a light emitting diode (LED).

In some embodiments of the invention, imaging capsule 110 has a diameter at least twice or even four times as large as the diameter of inner shaft 148. Possibly, imaging capsule 110 has a diameter at least six or even at least eight time as large as the diameter of inner shaft 148.

Inner shaft 148 optionally has a varying stiffness over its length. In some embodiments of the invention, inner shaft 148 is most stiff at its proximal end and its stiffness decreases gradually along its length until it is very flexible at its distal end where it connects to imaging capsule 110. Optionally, the change in the stiffness is gradual, such that the stiffness changes continuously over the length of inner shaft 148. Alternatively or additionally, inner shaft 148 is formed of a plurality of segments, which have a same flexibility over their entire length. In an exemplary embodiment of the invention, inner shaft 148 includes four main segments, a very flexible segment 230, a flexible segment 232, a less flexible segment 234 and a stiff segment 236.

Elongate tube 102 optionally has similar flexibility characteristics to those of inner shaft 148. Alternatively, elongate tube 102 has a changing flexibility over its length, although with a different pattern than of inner shaft 148. In some embodiments of the invention, elongate tube 102 is less flexible than inner shaft 148, over most of its length.

### Operation

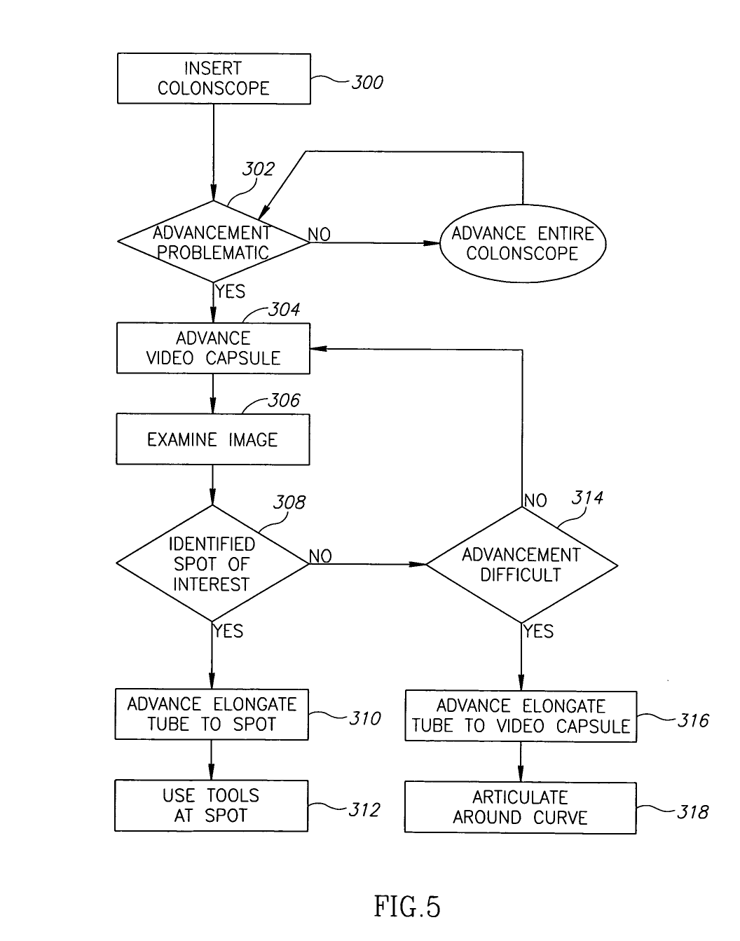

FIG. 5 is a flowchart of acts performed in a medical procedure using colonscope 100, in accordance with an exemplary embodiment of the invention. Elongate tube 102 is optionally inserted (300) into a patient with imaging capsule 110 within slot 106. When (302) the advancement of elongate tube 102 is problematic due to a narrowing in the patient's colon (e.g., when the sigmoid is reached), inner shaft 148 is advanced (304) ahead together with imaging capsule 110 on its distal end, while elongate tube 102 remains stationary. Images from imaging capsule 110 are optionally examined (306) when imaging capsule 110 is advanced in areas of interest of the colon. When a point of interest is identified (308) in the captured images, elongate tube 102 may be advanced (310) over inner shaft 148 to the location of imaging capsule 110, in order to allow usage (312) of tools within elongate tube 102 and/or in order to use working channel 118, at the point of interest.

In some embodiments of the invention, when (314) advancement of imaging capsule 110 is difficult or not possible due to a curve in the colon (e.g., at the splenic flexure, at the hepatic flexure), elongate tube 102 is advanced (316) over inner shaft 148 to imaging capsule 110. It is noted that the advancement of elongate tube 102 over inner shaft 148 is relatively fast, as inner shaft 148 serves as a guide-wire. At the curve, imaging capsule 110 enters into slot 106 and elongate tube 102, with imaging capsule 110 therein, is articulated (318) passed the curve, using articulation coil 138. Thereafter, imaging capsule 110 is optionally advanced (304) on its own, without elongate tube 102, until the next curve is reached.

Alternatively to identifying a curve due to difficulty in advancement, the advancement is performed under control of an imaging procedure (e.g., ultrasound), which allows identification of curves. Further alternatively, curves are identified in the view acquired by camera 202.

When it is desired to view the colon using the rear camera, elongate tube 102 is retracted, if necessary, in order to allow a sufficient field of view for the rear camera.

The advancement of elongate tube 102 toward imaging capsule 110 is optionally performed until slot 106 encompasses the capsule. Optionally, during the advancement of tube 102, capsule 110 remains stationary. Alternatively or additionally, tube 102 is advanced close to capsule 110 and the insertion of imaging capsule 110 into slot 106 is performed by retraction of the capsule into the slot. In some embodiments of the invention, control knob 164 can be used to rotate imaging capsule 110, so that it properly fits into slot 106. In some embodiments of the invention, an alignment marking 178 (FIG. 1) is positioned on the proximal end of elongate tube 102. A corresponding marking 182 is optionally located on control knob 164. When markings 178 and 182 are aligned, imaging capsule 110 can fit into slot 106.

In some embodiments of the invention, the colon is inspected during insertion of colonscope 100 into the patient. Alternatively, colonscope 100 is first inserted all the way into the patient and the inspection is performed as the colonscope is retracted from the patient. In other embodiments of the invention, the inspection is performed both during insertion and during removal from the patient.

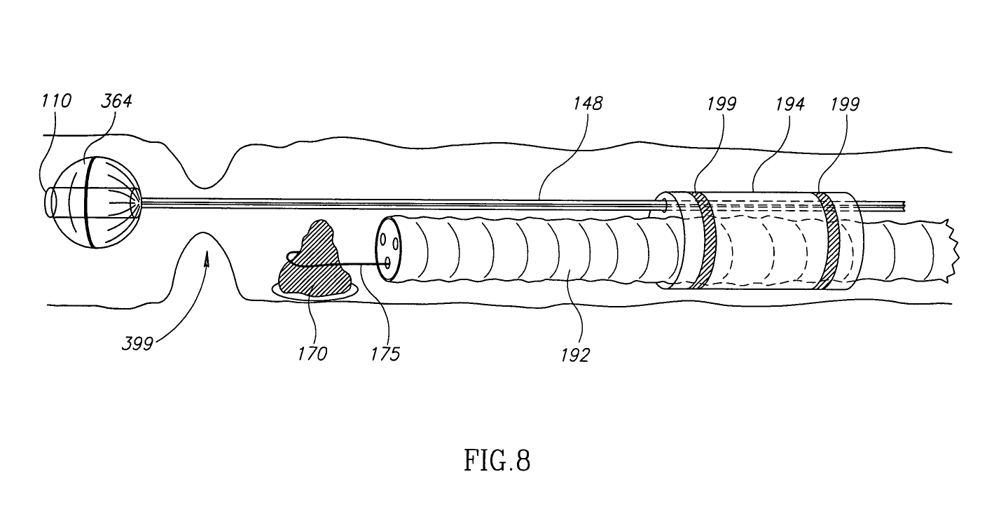

Optionally, if desired, elongate tube 102 may be removed from the patient while capsule 110 and inner shaft 148 remain in the patient, as described hereinbelow with reference to FIG. 8. For example, elongate tube 102 may connect to a polyp and be removed with the polyp. The reinsertion of elongate tube 102 over inner shaft 148 is expected to be faster than if it is reinserted without the leading of inner shaft 148. A large polyp may be removed in a sequence of a plurality of removals and reinsertions of elongate tube 102 which may be quite rapid due to the insertion of inner shaft 148. The removal of polyps is possibly much faster in this way, than if it needs to be removed through a working channel of elongate tube 102.

In some embodiments of the invention, after capsule 110 is in place, elongate tube 102 may be removed from the patient and another tool, may be inserted into the patient over inner shaft 148. For example, in accessing tissue beyond the ileocecal valve, it may be desired to use a narrow shaft (e.g., inner shaft 148) in advancing, rather than using a larger tool (e.g., elongate tube 102). Inner shaft 148 may be used in these embodiments to lead other tools to remote areas of the intestine.

In some embodiments of the invention, inner shaft 148 is used together with another shaft (e.g., a catheter) for advancing into remote areas of the intestine. Optionally, when one of the shafts gets stuck, it is anchored in place, for example by inflating a balloon, and the other shaft is advanced along its length and advanced beyond its distal end. In some embodiments of the invention, both the shafts carry imaging capsules at their distal ends. Alternatively, only inner shaft 148 carries an imaging capsule, while the other shaft does not carry an imaging head. Further alternatively or additionally, elongate tube 102 may be inserted into the patient with a balloon catheter not having a video capsule on its distal end. After the balloon catheter is anchored near the ileocecal valve, a catheter with imaging capsule 110 on its distal end is inserted into the patient over the balloon catheter. In some embodiments of the invention, advancement with two balloon catheters may be performed without an imaging capsule at all.

Alternatively to inserting imaging capsule 110 into the patient together with elongate tube 102, imaging capsule 110 is inserted into the patient through a channel (e.g., a working channel) of elongate tube 102, after elongate tube is in the patient. Further alternatively or additionally, imaging capsule 110 is first inserted into the patient and only afterwards is elongate tube 102 delivered along inner shaft 148.

### Radially Expandable Imaging Head

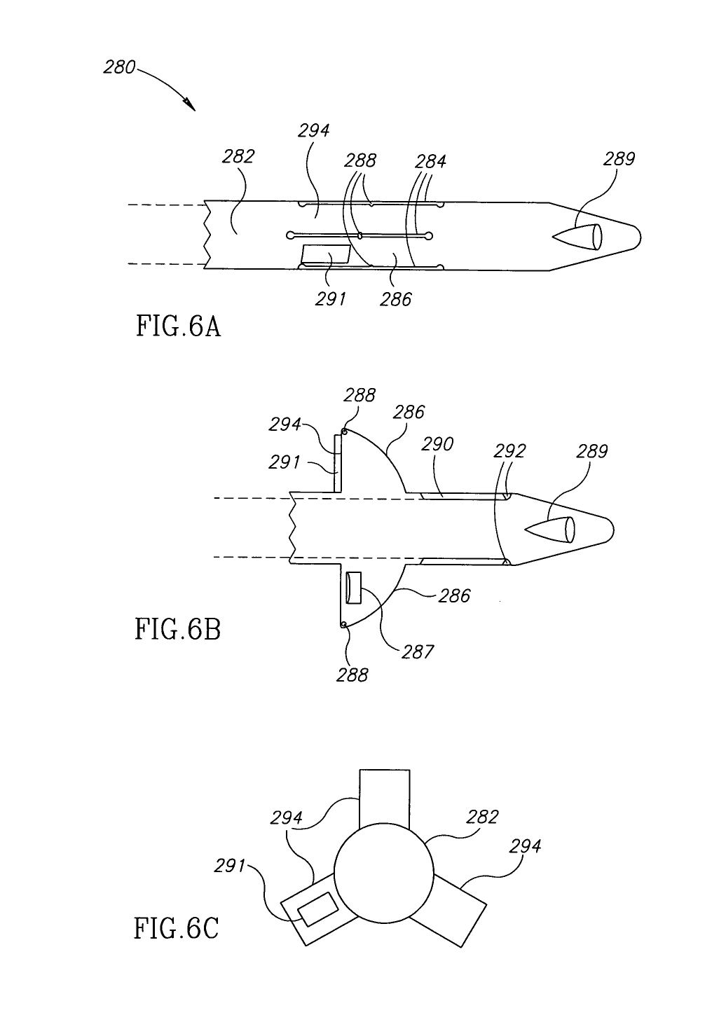

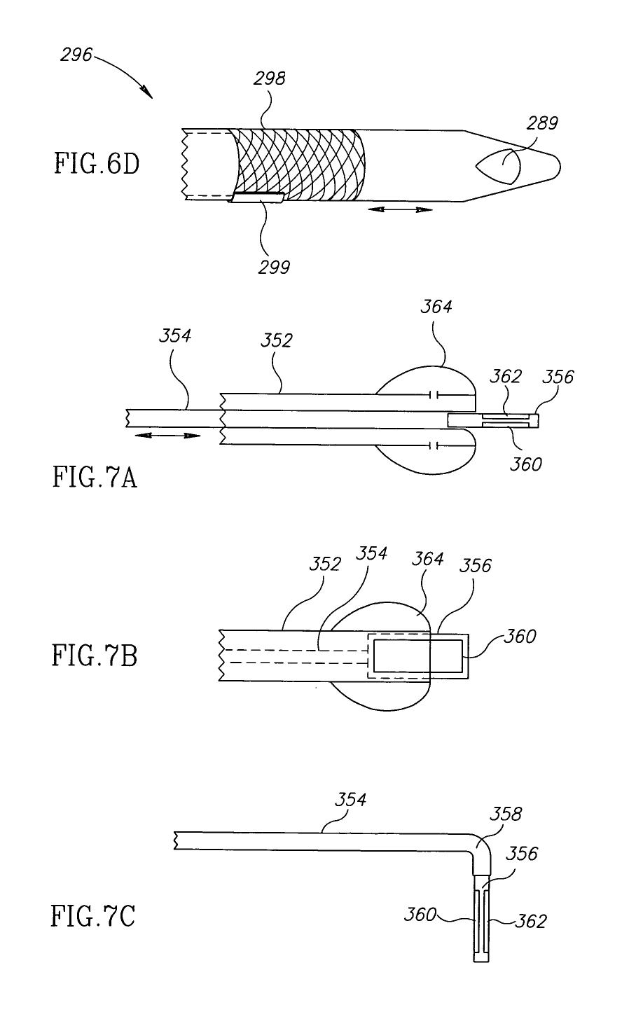

FIGS. 6A-6C are schematic illustrations of an imaging unit 280, in accordance with another exemplary embodiment of the invention. Imaging unit 280 comprises a tube 282 sized to pass through a channel of elongate tube 102 instead of inner shaft 148 and capsule 110. A plurality of slits 284 in tube 282 optionally define bendable strips 286. Preferred bend points 288 are optionally defined in strips 286, such that upon applying a proximally directed force on tube 282, strips 286 bend at bend points 282, as shown in FIG. 6B. In an exemplary embodiment of the invention, the proximally directed force is applied by pulling on an inner actuating tube 290 connected to tube 282 at one or more points distal of strips 286. Inner actuating tube 290 may optionally be used also to straighten strips 286, for example when desired to retract imaging unit 280 through the channel. Alternatively or additionally to inner actuating tube 290, other elements may be used to actuate the proximally directed force, for example one or more wires.

A forward view camera 289 is optionally mounted toward a distal end of tube 282. Alternatively or additionally, one or more of proximal portions 294 of bendable strips 286, proximal to bend points 288, carry a camera 291 (e.g., a CCD or CMOS strip) for backward view. Further alternatively or additionally, one or more of proximal portions 294 is transparent, possibly including a focusing lens, and has a camera 287 behind it. Optionally, the extent of bending of strips 286 is adjustable by a physician, such that proximal portions 294 may be positioned at an angle of 90° relative to tube 282, as shown in FIG. 6B, or at greater or smaller angles.

In some embodiments of the invention, tube 282 includes three bendable strips 286, as shown in FIG. 6C. Alternatively, tube 282 includes more than three strips 286 or fewer than three strips. In an exemplary embodiment of the invention, tube 282 has a diameter of about 3.5 millimeters and each of three strips 286 has a width of about 3.6 millimeters. Other dimensions may also be used, for example in which slits 284 are wider and cover at least 10% or even more of the circumference of tube 282.

FIG. 6D is an alternative implementation of a foldable tube 296, in accordance with an exemplary embodiment of the invention. Foldable tube 296 comprises instead of slits 284 and strips 286, an elastic braid 298, formed from a super elastic alloy or a plastic. Elastic braid 298 is foldable in manner similar to strips 286, to the orientation shown in FIG. 6B. One or more cameras 299 mounted on a proximal portion of the braid serves as for acquiring rear view images.

Tubes 282 and 296 are optionally rotatable within the patient, to allow freedom in directing images by the reflective surfaces toward imaging head 109.

FIGS. 7A and 7B are side and back views of an imaging unit 350, in accordance with an exemplary embodiment of the invention. Imaging unit 350 comprises an outer tube 352 and an inner tube 354, which is slidable relative to outer tube 352. At its distal end, inner tube 354 carries a camera sheet 356 which can be held within the outer tube or which can be extended out of outer tube 352. Camera sheet optionally carries a forward view camera 362 and/or a rear view camera 360.

FIG. 7C illustrates inner tube 354 with camera 356 in a rest state, in accordance with an exemplary embodiment of the invention. A distal portion of inner tube 354 optionally comprises a shape memory alloy wire or other elastic material. In its rest state, inner tube 354 optionally has a 90° bend at a point close to the connection with camera 356. When inner tube 354 is retracted into outer tube 352, camera 356 axially extends with outer tube 352, such that imaging unit 350 has a small cross section, for example with a diameter of less than 3.5 millimeters, such that it may pass through a working channel of an endoscope. When, however, inner tube 354 is extended out of outer tube 352, cameras 360 and/or 362 are extended to acquire images. It is noted that inner tube 354 may be only partially extended out of outer tube 352, so as to direct the cameras at any of a large span of angles as required.

In some embodiments of the invention, outer tube 352 carries a balloon 364 which may be inflated through the interior of tube 352 or a channel passing therethrough. The inflating of balloon 364 may be used to anchor imaging unit 350 within the patient, for anchoring, for example when imaging unit 350 additionally serves as a guidewire. During insertion, balloon 364 is deflated to allow fast insertion. When a desired location is reached, balloon 364 is optionally inflated to achieve anchoring of the balloon in place.

It is noted that a balloon may be mounted on any of the imaging units described above, including on imaging capsule 110. A balloon mounted on imaging capsule 110 is optionally inflated through inner shaft 148. The balloon optionally comprises a disc shaped balloon which expands radially from the outer surface of imaging capsule 110. Alternatively, imaging capsule 110 is implemented substantially entirely by a balloon, such that the inflation of the balloon changes the orientation of the imaging direction of the capsule.

FIG. 8 is a schematic illustration of use of imaging capsule 110 and inner shaft 148 as a monorail for an insertion tube 192 of an endoscope, in accordance with an exemplary embodiment of the invention. Although inner shaft 148 could be used as a monorail for a dedicated colonscope, such as colonscope 100, FIG. 8 illustrates its use with a conventional endoscope not adapted for use with inner shaft 148.

After imaging capsule 110 is properly positioned, for example beyond the ileocecal valve 399, balloon 364 is inflated. If inserted with an endoscope, the endoscope may be removed from the patient. A sleeve 194 is optionally mounted onto endoscope 192, for example using one or more bands 196. A narrow channel 188 in sleeve 194 is mounted on inner shaft 148 allowing fast insertion of endoscope 192 over shaft 148 into the patient. In order to remove a polyp 170, a snare 175 is optionally extended out of endoscope 192 and attached to the polyp. Endoscope 192 is then removed from the patient along inner shaft 148. This process may be repeated several times in order to remove a number of polyps or a large polyp which requires dissection for removal.

Sleeve 194 optionally extends over a short length of endoscope 192, for example less than 10 centimeters or even less than 2 centimeters. Alternatively, the sleeve extends over a substantial length of endoscope 192, for example more than half a meter, or even close to the entire length of endoscope 192.

Capsule with Threading

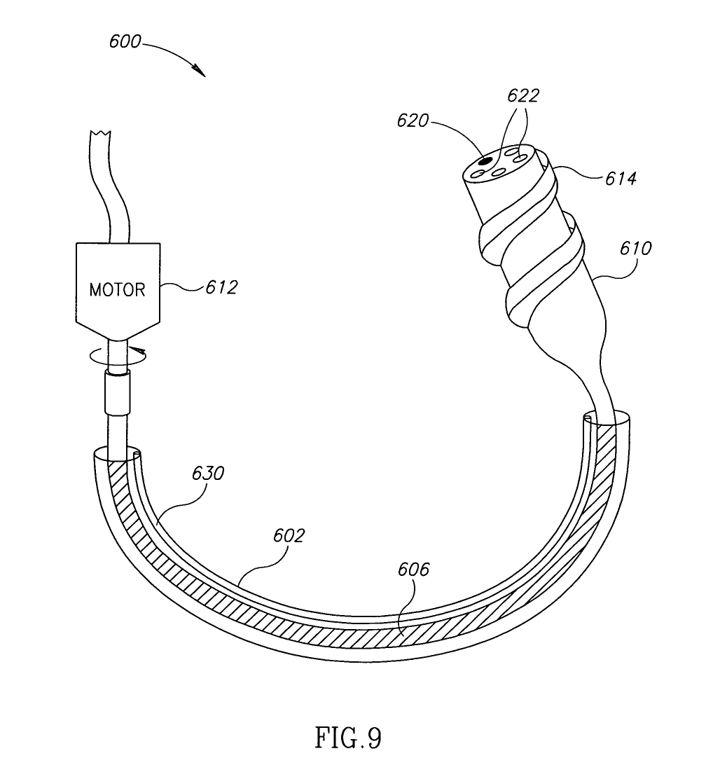

FIG. 9 is a schematic illustration of a colonscope 600, in accordance with an exemplary embodiment of the invention. Colonscope 600 comprises an external tube 602 and an imaging capsule 610, which is rotatable relative to the external tube. An internal shaft 606 connects capsule 610 to a motor 612, such that rotation force from motor 612 rotates internal shaft 606, which rotates capsule 610. An outer surface of imaging capsule 610 is optionally covered by a threading 614, which causes the imaging capsule to advance or retract along the colon (or other internal body channel) when imaging capsule 610 is rotated. Optionally, threading 614 has a helical form.

Capsule 610 optionally includes on its distal end, a camera 620 and one or more (e.g., three) light sources 622.

As shown, capsule 610 has a larger diameter than external tube 602, such that the external tube follows capsule 610 into the patient relatively easily. In some embodiments of the invention, external tube 602 isolates the rotation of internal shaft 606 from tissue of the patient, so that friction between the internal shaft and body tissue does not dampen the rotation. Optionally, external tube 602 defines one or more working and/or fluid channels 630 adapted to lead tools and/or fluids to a distal end of the tube. Alternatively to capsule 610 having a larger diameter than external tube 602, capsule 610 has a diameter which is not larger than the diameter of external tube 602, for example similar to that shown in FIG. 1. In fact, capsule 110 of FIG. 1 has a threading on it to facilitate its advancement.

Optionally, threading 614 comprises a plastic extrusion deposited on the imaging capsule. In some embodiments of the invention, the threading 614 is rigid, so as to maximize the advancement force it applies. In some embodiments of the invention, the threading includes at least two turns or even at least four turns around imaging capsule 610. Alternatively, the threading 614 includes a large number of turns, for example at least 10 or at least 20 turns.

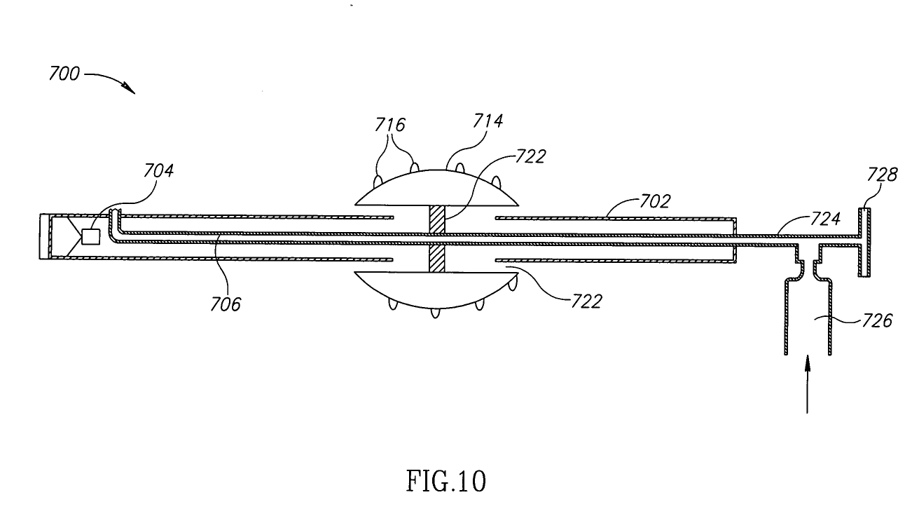

FIG. 10 is a schematic cross sectional view of an endoscope 700, in accordance with another exemplary embodiment of the invention. Endoscope 700 comprises an external tube 702 having a camera 704 at a distal end thereof. A working channel 706 optionally serves to lead fluids to a distal end of endoscope 700 and/or to apply suction to the distal end of the endoscope. In addition, working channel 706 is optionally connected, for example through radial bars 722, to an outer threaded shield 714 with threading 716 along the length of external tube 702. Rotation of working channel 706 while endoscope 700 is within a narrow body passage, causes shield 714 to rotate, such that threading 716 advances within an internal body lumen relative to the body tissue and causes endoscope 700 to advance within the patient.

Working channel 706 is shown as not reaching the end of external tube 702, but rather protrudes from the side of external tube 702 before the distal end of endoscope 700. Alternatively, working channel 706 extends until the distal end of endoscope 700. In accordance with this alternative, camera 704 is optionally positioned on an outer annular area of external tube 702, so as not interfere with channel 706.

In some embodiments of the invention, working channel 706 connects at its proximal end 724 to a fluid source 726. In addition, proximal end 724 optionally connects to a handle 728, which is used to rotate working channel 706. In some embodiments of the invention, handle 728 is rotated manually by a physician. Alternatively or additionally, handle 728 is coupled to a motor which rotates the handle.

As shown in FIG. 10, threading 716 is located about half way along the length of endoscope 700. In other embodiments of the invention, threading 716 is located closer to the distal end of external tube 702.

In some embodiments of the invention, the threading covers only a small portion of the axial length of endoscope 700, for example less than 20%, less than 10% or even less than 5% of the length of the endoscope. Alternatively, the threading may cover a substantial length of endoscope 700, for example at least 25% or even at least 30%.

While imaging capsule 110 is described as having a camera mounted thereon, in some embodiments of the invention, images are acquired by the imaging capsule through a bundle of optical fibers which lead the images they collect to a proximal portion of the endoscope, where they are displayed or captured by a proximal camera.

It will be appreciated that the above-described apparatus and methods may be varied in many ways, including, varying sizes, shapes and materials used. It should also be appreciated that the above described description of methods and apparatus are to be interpreted as including apparatus for carrying out the methods, and methods of using the apparatus.

The present invention has been described using non-limiting detailed descriptions of embodiments thereof that are provided by way of example and are not intended to limit the scope of the invention. For example, although some embodiments are described with relation to a colonscope, the features of these embodiments may be used with other medical tubes, such as other endoscopes, catheters and trocars. It should be understood that features and/or steps described with respect to one embodiment may be used with other embodiments and that not all embodiments of the invention have all of the features and/or steps shown in a particular figure or described with respect to one of the embodiments. Variations of embodiments described will occur to persons of the art. Furthermore, the terms “comprise,” “include,” “have” and their conjugates, shall mean, when used in the claims, “including but not necessarily limited to.”

It is noted that some of the above described embodiments may describe the best mode contemplated by the inventors and therefore may include structure, acts or details of structures and acts that may not be essential to the invention and which are described as examples. Structure and acts described herein are replaceable by equivalents which perform the same function, even if the structure or acts are different, as known in the art. Therefore, the scope of the invention is limited only by the elements and limitations as used in the claims.

1. An endoscope, comprising:
an outer elongate tube;
an inner shaft that runs through the outer elongate tube and can be advanced independently of the outer tube; and
an imaging capsule including at least one image capturing unit, mounted on a distal end of the inner shaft, the imaging capsule having a largest cross-section area smaller than the cross section area of the outer elongate tube.

2. An endoscope according to claim 1, wherein the outer tube comprises a slot adapted to receive at least half of the imaging capsule, on its distal end.

3. An endoscope according to claim 2, wherein the slot is adapted to receive the imaging capsule in its entirety.

4. An endoscope according to claim 2, wherein when the outer tube includes at least one channel adapted to direct fluids at the imaging capsule, when the imaging capsule is in the slot.

5. An endoscope according to claim 1, wherein the outer tube comprises an articulation section which can be controllably articulated.

6. An endoscope according to claim 5, wherein the inner shaft does not have a mechanism for controllable articulation.

7. An endoscope according to claim 1, wherein the inner shaft has different levels of flexibility over its length.

8. An endoscope according to claim 7, wherein the inner shaft has a flexibility which decreases toward the imaging capsule.

9. An endoscope according to claim 1, wherein the image capturing unit comprises a CCD.

10. An endoscope according to claim 1, wherein the imaging capsule comprises at least one image capturing unit with a view directed proximally toward the outer tube.

11. An endoscope according to claim 1, wherein the imaging capsule has a largest diameter at least twice the diameter of the inner shaft.

12. An endoscope according to claim 1, wherein the outer elongate tube defines at least one working channel running along its length.

13. An endoscope according to claim 1, wherein an extent of axial movement of the inner elongate tube within the outer elongate tube is limited.

14. An endoscope according to claim 1, wherein the imaging capsule has a larger cross section than the inner elongate tube, which prevents it from being retracted proximally through the outer elongate tube.

15. An endoscope according to claim 1, wherein the imaging capsule is adapted to pass through a channel of the outer tube.

16. An endoscope according to claim 1, wherein the imaging capsule has a collapsed state in which it can pass through a channel of the outer tube and an expanded state in which it cannot pass through a channel of the outer tube.

17. An endoscope according to claim 1, wherein an axial extent of a portion of the imaging capsule having a cross-section area larger than a cross section area of the inner shaft is greater than a diameter of the imaging capsule.

18. An endoscope according to claim 1, wherein an axial extent from a most proximal camera of the imaging capsule to its distal end is greater than a diameter of the imaging capsule.

19. An endoscope according to claim 1, wherein the imaging capsule has a cross-section diameter of less than 3.5 millimeters.

20. An endoscope according to claim 1, wherein the imaging capsule comprises a balloon.

21. An endoscope according to claim 20, wherein the balloon is adapted to expand radially, without displacing an imaging direction of the imaging capsule.

22. An invasive medical probe, comprising:
a first portion comprising a tube defining an outer surface adapted to contact internal body tissue;
a second portion comprising an outer surface adapted to contact internal body tissue, the second portion being adapted to rotate relative to the first portion; and
a helical threading on the outer surface of the second portion, adapted to advance the second portion within a body cavity when the second portion is rotated relative to the body portion.

23. A probe according to claim 22, comprising a motor adapted to rotate the second portion.

24. A probe according to claim 22, wherein the second portion is close to a distal end of the medical probe.

25. A probe according to claim 22, comprising an internal shaft extending through the first portion and connecting the second portion to a proximal end of the medical probe.

26. A method of advancing an endoscope within a patient, comprising:
inserting a shaft having an imaging capsule at its distal end to a body lumen of a patient; and
advancing an outer tube, having a cross section larger than the cross section of the shaft, over the shaft, into the body lumen.

27. A method according to claim 26, wherein the imaging capsule has a cross section area greater than the cross section area of the shaft.

28. A method according to claim 26, wherein the imaging capsule has a cross section area substantially equal to the cross section area of the shaft.

29. A method according to claim 26, wherein the outer tube comprises an endoscope.

30. A method according to claim 26, wherein the outer tube comprises an articulation system and the shaft does not comprise an articulation system.

31. A method according to claim 26, wherein the imaging capsule comprises at least one camera mounted thereon.

32. A method according to claim 26, wherein inserting the shaft comprises inserting together with the outer tube.

33. A method according to claim 26, wherein inserting the shaft comprises inserting through a channel of the outer tube, after the outer tube was inserted separately into the patient.

34. A method according to claim 26, wherein advancing the outer tube over the shaft comprises advancing the outer tube with a channel of the outer tube riding over the shaft.

35. A method according to claim 26, wherein advancing the outer tube over the shaft comprises connecting a sleeve to the outer tube and passing the sleeve over the shaft.
